(19) United States
(12) Patent Application Publication
(10) Pub. No.: US 2025/0216014 A1
(43) Pub. Date: Jul. 3, 2025

(54) SEPARABLE BI-DIRECTIONAL CHECK VALVE ASSEMBLY

(71) Applicant: Luminoah, Inc., Charlottesville, VA (US)

(72) Inventors: J. Landon Gilkey, Charlottesville, VA (US); Hill Johnson, Charlottesville, VA (US); Joseph Neal Piper, Charlottesville, VA (US)

(21) Appl. No.: 18/990,320

(22) Filed: Dec. 20, 2024

Related U.S. Application Data
(60) Provisional application No. 63/615,407, filed on Dec. 28, 2023.

(51) Int. Cl. F16L 37/32; A61M 39/10

(52) U.S. Cl. CPC F16L 37/32; A61M 39/10; A61M 2039/1061; F16L 2201/20

(57) ABSTRACT

A breakaway coupling for use in fluid delivery systems comprises either a mechanical or magnetic coupling configuration. In the first embodiment, the coupling includes a plunger assembly having an inlet, a plunger chamber in fluid communication with the inlet, a stem chamber in fluid communication with the plunger chamber, and a plunger configured to move between positions. The first embodiment further includes a valve assembly having a valve chamber with a valve and rib, and an outlet. In the second embodiment, the coupling includes a first assembly having an inlet port, a first valve chamber housing a one-way valve, and a magnetic ring, and a second assembly having an outlet port, a second valve chamber, a protrusion, and a complementary magnetic ring. Both embodiments automatically separate and seal upon application of force.

### CROSS-REFERENCE TO RELATED APPLICATIONS

The present application claims priority to U.S. Provisional Application No. 63/615,407, filed Dec. 28, 2023, which is incorporated herein by reference in its entirety.

### TECHNICAL FIELD

The present disclosure relates to breakaway couplings for use with medical fluids. In particular, the present disclosure relates to a breakaway coupling assembly for use with enteral nutrition therapy that is configured to prevent the undesired flow of fluids when disconnected.

### BACKGROUND

Gastronomy button dislodgement is a common problem for patients requiring tube feeding. If a dislodgement occurs in the first six weeks after placement, an emergency procedure is required. Accordingly, a need exists for an improved breakaway coupling for use with enteral nutrition therapy.

### SUMMARY

The present disclosure provides, in at least one aspect, couplings comprising a plunger assembly comprising an inlet, a plunger chamber in fluid communication with the inlet, a stem chamber in fluid communication with the plunger chamber, and a plunger that moves from a first position to a second position, wherein, the plunger prevents the movement of a fluid between the plunger chamber and the stem chamber when in the first position, a valve assembly comprising, a valve chamber with a valve and a rib disposed within; and an outlet in fluid communication with the valve chamber, wherein, the coupling is moves between a connected state, where the two assemblies are joined together, and a disconnected state, where the two assemblies are disconnected, wherein, when the coupling is in its connected state, the rib retains the plunger in its second position, the stem chamber is in fluid communication with the valve chamber, and the fluid is able to flow between the inlet and the outlet and wherein, when the coupling is in its disconnected state, the fluid biases the plunger in its first position, preventing the flow of the fluid between the plunger chamber and the stem chamber.

In some embodiments, the plunger assembly further comprises a gasket disposed within a channel, fluidically seals the two assemblies when the coupling is in its connected state.

In some embodiments, the coupling further comprises a set of enteral feeding connectors respectively positioned at the inlet and outlet.

In some embodiments, the plunger further comprises a head and stem, wherein, the head is at least partially retained within the plunger chamber and the stem is at least partially retained within the stem chamber.

In some embodiments, the valve further comprises a diaphragm and slit, wherein, the plunger assembly pierces the diaphragm and retain the valve in an open position.

In some embodiments, the plunger further comprises a cutout sized and allows the fluid to pass along the stem.

In some embodiments, the plunger assembly further comprises a plunger outer housing and the valve assembly further comprises a valve outer housing, wherein the plunger outer housing is partially disposed within the valve outer housing when in the connected state.

In some embodiments, the plunger chamber comprises a first inner surface and a second inner surface forming circular faces of the chamber, and wherein the first inner surface and the second inner surface each comprise a respective circular hole.

In some embodiments, the plunger abuts against the second inner surface in the first position to prevent the fluid flow.

In some embodiments, the valve chamber comprises a first portion having a first diameter and a second portion having a second diameter different from the first diameter, the first and second portions being connected by a conical transitional section.

In some embodiments, the rib bisects the valve chamber and is sized to allow the fluid to flow past the rib toward the outlet.

In some embodiments, the plunger assembly and valve assembly are formed from a material selected from the group consisting of resin, plastic, metal, and composites.

In some embodiments, the plunger assembly and valve assembly automatically separate upon application of a predetermined force to prevent patient harm.

In some embodiments, the valve automatically close when the coupling transitions to the disconnected state to prevent unwanted fluid discharge.

The present disclosure provides, in at least another aspect, couplings comprising a first assembly comprising an inlet port, a first valve chamber housing a first one-way valve formed of a compliant material, and a first magnetic ring circumferentially disposed about the first assembly, a second assembly comprising, an outlet port, a second valve chamber housing a second one-way valve, a protrusion extending from the second assembly toward the first assembly, and a second magnetic ring circumferentially disposed about the second assembly, wherein, the coupling moves between a connected state, where the two assemblies are magnetically joined together, and a disconnected state, wherein, when the coupling is in its connected state, the protrusion mechanically engages with and displaces the first one-way valve creating a continuous fluid pathway between the inlet port and the outlet port, and wherein, when the coupling is in its disconnected state, the first one-way valve automatically returns to a sealed position preventing fluid discharge.

In some embodiments, the first and second magnetic rings are oriented with opposite polarities to create an attractive force when the assemblies are brought into proximity.

In some embodiments, the first valve chamber and second valve chamber are generally cylindrical in shape with diameters sufficient to accommodate their respective valves.

In some embodiments, the first one-way valve is oriented to permit fluid flow toward the second assembly while preventing backflow toward the inlet port, and the second one-way valve is oriented to prevent backflow from the outlet port.

In some embodiments, the magnetic rings separate when a predetermined tensile force is applied to prevent patient harm.

Other aspects of the disclosure will become apparent by consideration of the detailed description and accompanying drawings.

### BRIEF DESCRIPTION OF THE DRAWINGS

FIG. 1 is a perspective view of a wearable fluid delivery system including a coupling shown in a connected state.

FIG. 2 is a perspective view of the coupling removed from the fluid delivery system of FIG. 1, shown in a connected state.

FIG. 3 is a perspective cross-sectional view of the coupling of FIG. 2, shown in a connected state.

FIG. 4 is a perspective view of the coupling of FIG. 2, shown in a disconnected state.

FIG. 5 is a perspective view of a cross section through the coupling of FIG. 2, shown in a disconnected state.

FIG. 6 is a perspective view of the second embodiment of the coupling using a pair of one-way valves and magnetic coupling elements, shown in a connected state.

FIG. 7 is a perspective view of the coupling of FIG. 6, the two assemblies shown in a disconnected state.

FIG. 8 is a cross-sectional view of the coupling of FIG. 6, the two assemblies shown in a connected state.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Gastronomy button dislodgement is a common problem for patients requiring tube feeding. If a dislodgement occurs in the first six weeks after placement, an emergency procedure is required. Accordingly, the need exists for a separable bi-directional check valve assembly placed in-line with a nutrient administration system that would allow the assembly to automatically separate upon the application of force to prevent patient harm. Such assembly would include features to prevent unwanted discharge of nutrition from the nutrient administration system upon separation. An example nutrient administration system, or fluid delivery system, is described in U.S. patent application Ser. No. 18/446,039, filed Aug. 8, 2023, the entire contents of which are incorporated herein by reference.

### 1. Definitions

The terms “substantially” or “generally” are used to provide flexibility by recognizing that a given characteristic need not be perfectly embodied to have the desired result. Those of ordinary skill in the art will recognize that many characteristics described herein may be essentially present without strict adherence to the characteristic's definition.

The term “coupled,” as used herein, is defined as “connected,” although not necessarily directly, and not necessarily mechanically. The term coupled is to be understood to mean physically, magnetically, chemically, fluidly, electrically, or otherwise coupled, connected or linked and does not exclude the presence of intermediate elements between the coupled elements absent specific contrary language.

As used herein, the term “subject” or “patient” refers to any animal (e.g., a mammal), including, but not limited to, humans, non-human primates, companion animals, livestock, equines, rodents, and the like, which is to be the recipient of a particular treatment. Typically, the terms “subject” and “patient” are used interchangeably herein in reference to a human subject.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising.” “consisting of” and “consisting essentially of,” the embodiments or elements presented herein, whether explicitly set forth or not.

In the foregoing description of preferred embodiments, specific terminology has been resorted to for the sake of clarity. However, the invention is not intended to be limited to the specific terms so selected, and it is to be understood that each specific term includes all technical equivalents which operate in a similar manner to accomplish a similar technical purpose. Terms such as “top” and “bottom”, “front” and “rear”, “inner” and “outer”, “above”, “below”, “upper”, “lower”, “vertical”, “horizontal”, “upright” and the like are used as words of convenience to provide reference points.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Description

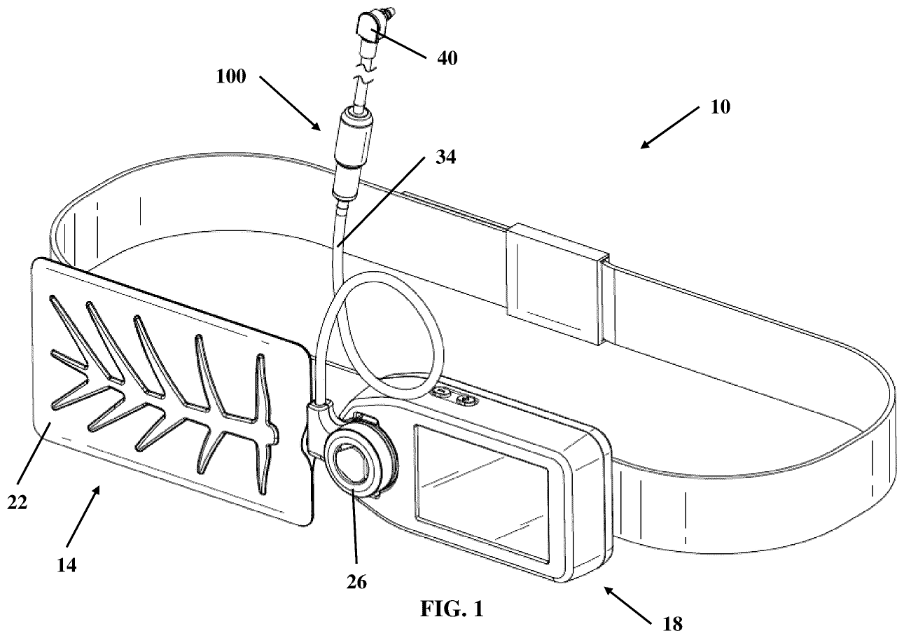

An exemplary fluid delivery system 10 is provided in FIG. 1. In some embodiments, the wearable fluid delivery system 10 is portable and utilized for intermittent infusions, continuous infusions, or night feeds.

System 10 includes a pouch assembly 14, which is a single-use device (e.g., a single use disposable), and a module 18, which is reusable. System 10 includes disposable elements (e.g., the disposable pouch assembly 14) that may be replaced after each feeding, for example. The pouch assembly 14 includes a nutrition container 22 with a fluid (e.g., a fluid reservoir) contained within. In some embodiments, the fluid is a nutrient fluid, a medication, or other therapeutic fluid, that is pumped from nutrition container 22 for delivery to the user.

With continued reference to FIG. 1, the pouch assembly 14 also includes a pump 26, a tube 34, a coupling 100, and an end connector 40. Tube 34 is coupled to pump 26 and the end connector 40. Pump 26 is coupled to the nutrition container 22 and the tube 34, and operation of the pump 26 causes the fluid in the nutrient container 22 to travel from nutrient container 22, through the tube 34 and coupling 100, to the end connector 40.

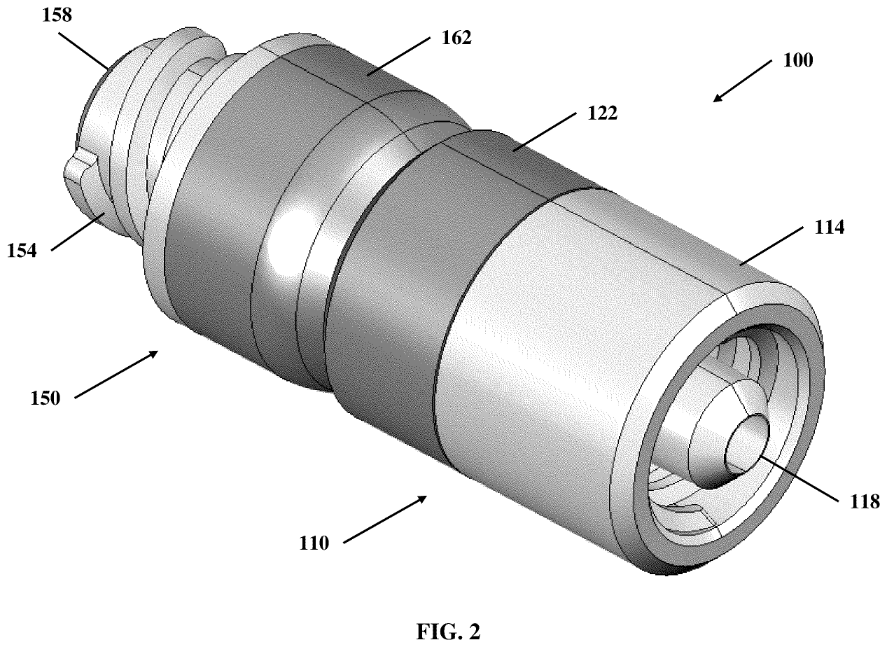

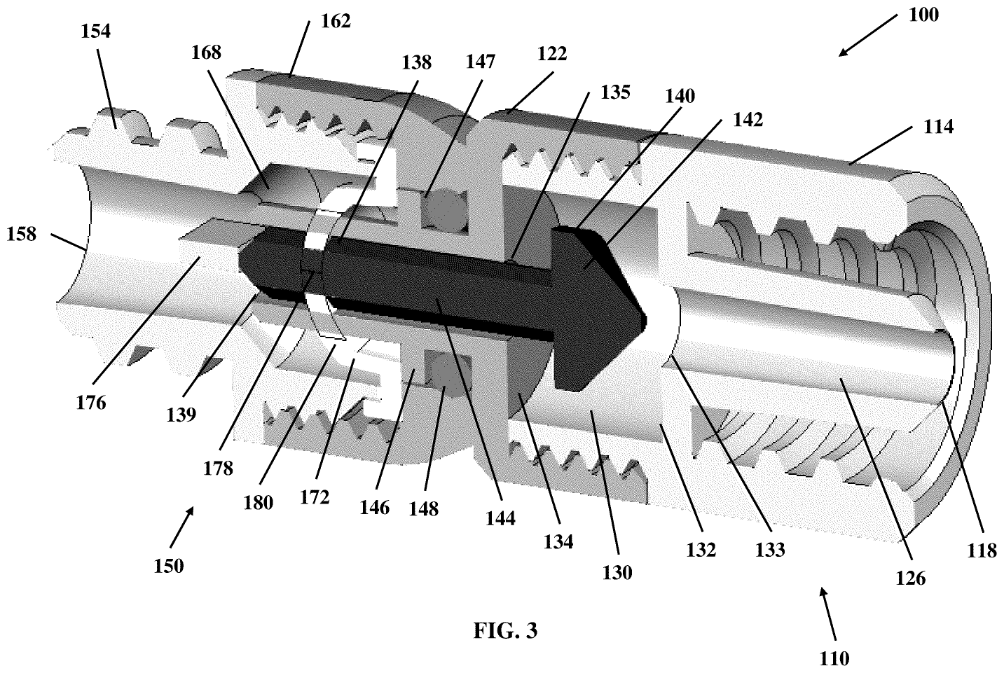

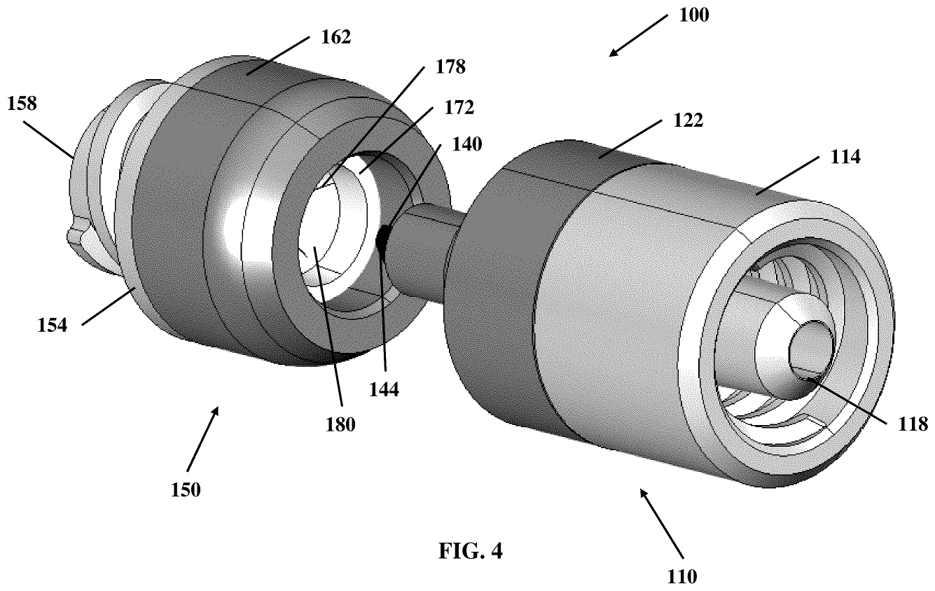

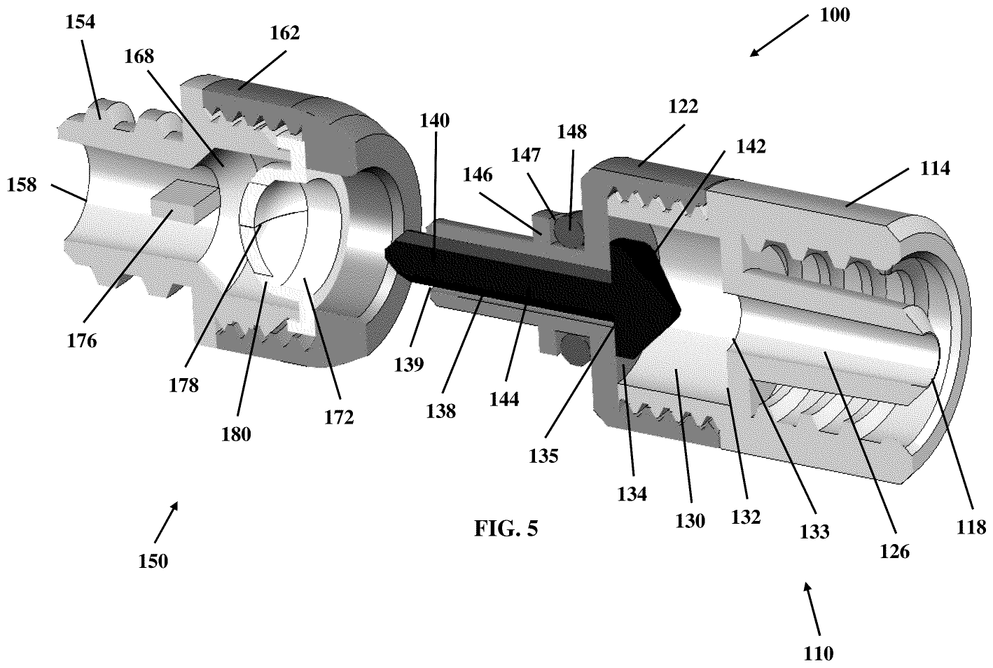

With reference to FIG. 2, provided herein is the coupling 100 in a connected state. Coupling 100 is comprised of two assemblies, a plunger assembly 110 and a valve assembly 150 that are configured to be joined together in a connected state (FIGS. 2-3) and separated from each other in a disconnected state (FIGS. 4-5). In some embodiments, coupling 100 is positioned in line with the tube 34 and oriented so that the plunger assembly 110 is closer to the pump 26 while the valve assembly 150 is positioned closer to the end connector 40.

With continued reference to FIG. 2, the plunger assembly 110 includes a first tube connector 114 with an inlet port 118 located at its proximal end that when connected to the system 10 of FIG. 1 is in fluid communication with the tube 34 and pump 26. The plunger assembly 110 also includes a plunger outer housing 122 that interfaces with the valve assembly 150. The tube connector 114 includes internal threading and bevel configured to interface with industry standard enteral feeding connectors (e.g., ENFit® connectors). Similarly, the valve assembly 150 includes a second tube connector 154 with an outlet port 158 that when connected to the system of FIG. 1, is in fluid communication with the end connector 40, outer threading configured to interface with standard enteral feeding connectors, and an valve outer housing 162 that interfaces with the plunger assembly 110. The tube connectors (114, 154) and outer housings (122, 162) of both the plunger assembly 110 and the valve assembly 150 may be formed from resin, plastics, metals, composites, or other materials recognized by those of ordinary skill in the art as being suitable for the disclosed purpose and may be formed using injection molding, machining, additive manufacturing, or other known processes.

With reference to FIG. 3, the coupling 100 is shown in cross section. The first tube housing 114 includes an inner wall forming an inlet chamber 126, cylindrical in shape with a length greater than its diameter, extending axially from the inlet 118 to the plunger chamber 130. The plunger chamber 130 is also generally cylindrical in shape and includes a first inner surface 132 and an opposite second inner surface 134 forming the circular faces of the cylindrical chamber. The second inner surface 134 is formed by the plunger outer housing 122. Each inner surface has a respective circular hole, the first circular hole 133 connects the inlet chamber 126 with the plunger chamber 130 and the second circular hole 135 connects the plunger chamber 130 to the stem chamber 138 formed by the outer housing 122, and an opening 139 at the distal end of the stem chamber 138. It is through these series of holes and chambers that fluid would flow through the plunger assembly 110 and to the valve assembly 150.

The plunger assembly 110 further includes a plunger 140 with a head 142 and stem 144 positioned along the coupling's central axis. The head of plunger 142 is retained inside the plunger chamber 130 and is generally conical in shape, with the flat end facing the second inner surface 134 of the chamber 130. The stem 144 extends from the plunger head 142 into the stem chamber 138. The stem 144 is generally cylindrical in shape and has an outer diameter smaller than the inner diameter of the stem chamber 138 to allow for fluid to pass between the stem 144 and the outer housing 122. In some embodiments, the stem 144 includes a notch or cutout to facilitate fluid flow along the stem 144. Ridge 146 circumscribes the external surface of the outer housing 122 forming a channel 147. Nested inside channel 147 is an O-ring 148 or gasket, positioned and configured to seal the mating surfaces between the two assemblies (110, 150) and prevent fluid from escaping the coupling when in a connected state and in operation.

The tube connector 154 of the valve assembly 150 includes a hollow valve chamber 168 with two portions of different diameter connected by a conical transitional section. The larger diameter section houses a valve 172, a portion of the plunger outer housing 122 and stem 144 when in a connected state. The section of smaller diameter forms the coupling outlet 158 and contains a rib 176 bisecting the valve chamber 168. Rib 176 is sized appropriately to allow for fluid to flow pass the rib 176 and through the valve chamber 168 in the direction of the outlet 158. In some embodiments the valve 172 takes the form of a dome shaped diaphragm formed of sufficiently pliable material, with a slit 178 formed perpendicular to the coupling's major axis (shown more clearly in FIG. 5). In some embodiments, valve 172 behaves like a check valve and only allows fluid to flow in one direction when closed. Valve 172 is secured between the valve tube connector 154 and valve outer housing 162.

Still referencing FIG. 3, the outer housings (122, 162) of the two assemblies (110, 150) are mated to their respective tube connectors (114, 154) using a set of interlocking threads. The use of threads in this instance is to allow for easy disassembly and cleaning, but more permanent methods to mate the two components may be used including adhesives, soldering, interference fit, or any other known methods.

Referring to FIGS. 2-3, in combination with FIG. 1, in the coupling's connected state, fluid is able to pass from the pump 26, through the tube 34 and coupling 100, to the end connector 40. In the connected state, the plunger outer housing 122 is partially disposed inside the valve outer housing 162, so that the stem 144 extends into the valve chamber 168 and pierces through the slit 178 formed the valve's domed diaphragm (FIG. 3). Rib 176 is positioned and configured to push against stem 144 and prevent the steam head 142 from abutting the second inner surface 134 of the plunger chamber 130. By doing so, fluid is able to pass between the plunger stem 144 and walls of the stem chamber 138. Similarly, the plunger outer housing 122 prevents valve 172 from closing, allowing fluid to pass from the stem chamber 138 into the valve chamber 168.

In the coupling's connected state, fluid is able to pass from the pump 26, through the tube 34, and into the inlet 118 of the coupling 100. From there, fluid moves through inlet chamber 126, passed the plunger head 142 in the plunger chamber 130, and into the stem chamber 138. Fluid then continues along the plunger steam 144 and into valve chamber 168, where it flows past the rib 176 and continues to the outlet 158 where it reenters the tube 34.

Referring to FIGS. 4-5, the coupling is shown in its disconnected state with the plunger assembly 110 and valve assembly 150 detached from each other. Coupling 100 is designed to move from the connected state to the disconnected state when a sudden force is applied to the tube 34 or coupling 100 during operation. This is typically caused by the tube 34 accidentally becoming caught on an external object or the patient themselves. Coupling 100 also allows for a patient or another individual to intentionally detach the two assemblies (110, 150), such as in cases where the tube 34 needs to be untangled, or if system 10 malfunctions. In this state, the plunger 140 and valve 172 both function to prevent unwanted discharge of fluid from their respective assemblies (110, 150).

Referring now to FIGS. 4-5 in combination with FIG. 3. When disconnected, the coupling 100 automatically seals due to fluid pressure and direction of fluid flow. As discussed above, fluid is actively pumped through coupling 100 and moves generally from the right-hand side of the coupling 100 to the left-hand side (when viewed in FIGS. 2-3). When fluid flows passed the plunger 140, it pushes against the conical head 142 and exerts a biasing force along the coupling's primary axis in the direction of flow (right to left). In its connected state, rib 176 prevents movement of the plunger 140 towards the outlet. However, when coupling 100 is in its disconnected state, this force pushes the head 142 axially toward the opening 139 until it abuts against the second inner surface 134 of the plunger chamber 130. This creates a seal between the inner surface 134 and the plunger head 142 and prevents fluid from flowing into the stem chamber 138 and discharging from the plunger assembly 110. Similarly, without the outer housing 122 of the plunger assembly preventing the valve 172 from closing, it returns to its biased closed position. Any fluid remaining in valve chamber 168 would exert a pressure force against the convex surface of the valve's diaphragm 180 which only works to further bias the valve 172 to its closed position.

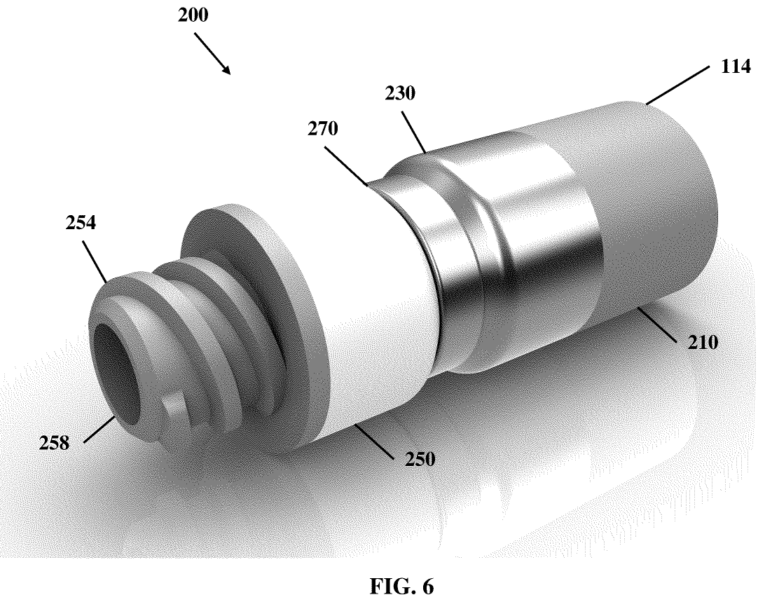

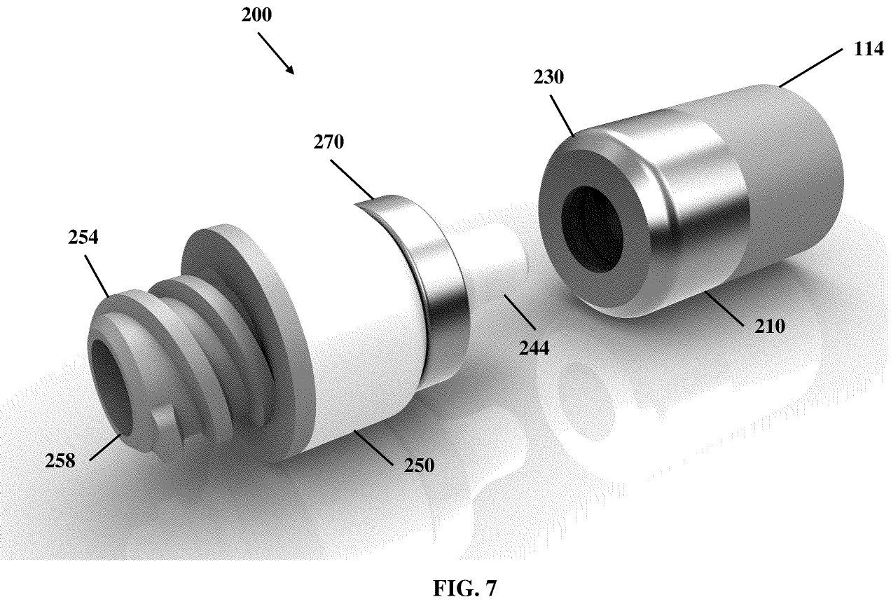

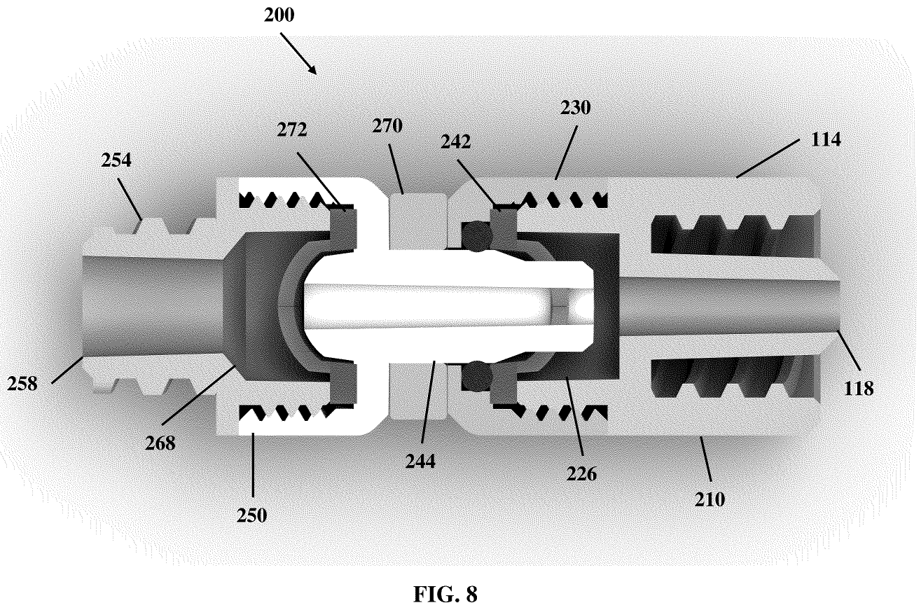

Referring now to FIGS. 6-8, coupling 200 comprises a first assembly 210 and a second assembly 250 that are configured to be magnetically joined together. The coupling can transition between a connected state (as shown in the perspective view of FIG. 6 and cross-sectional view of FIG. 8) and a disconnected state (as shown in the perspective view of FIG. 7). Similar to the first embodiment, coupling 200 is positioned in line with tube 34 between pump 26 and end connector 40. The first assembly 210 includes a first tube connector 214 with an inlet port 218, while the second assembly 250 includes a second tube connector 254 with an outlet port 258 and a protrusion 244.

With particular reference to FIG. 8, the internal structure of both assemblies is shown in cross-section. The first assembly 210 includes a valve chamber 226 housing a first one-way valve 242. The first one-way valve 242 is formed from a compliant material and oriented to permit fluid flow in the direction of the second assembly 250 while preventing backflow toward inlet port 218 when in its sealed position. The second assembly 250 contains a corresponding valve chamber 268 housing a second one-way valve 272, along with the protrusion 244 that extends from the second assembly toward the first assembly. The second one-way valve 272 is oriented to prevent backflow from outlet port 258. Both valve chambers (226, 268) are generally cylindrical in shape with a diameter sufficient to accommodate their respective valves and fluid flow requirements.

As best shown in FIG. 8, when in the connected state, the protrusion 244 of the second assembly 250 extends into the first assembly 210 and mechanically engages with the first one-way valve 242, displacing it from its sealed position. This displacement creates a continuous fluid pathway through both assemblies. The second one-way valve 272 remains in its normal operating position, regulating flow toward the outlet port 258. During operation, fluid enters through inlet port 218 of the first assembly, passes through valve chamber 226, flows past the displaced first one-way valve 242, continues through the second one-way valve 272, moves through valve chamber 268, and finally exits through outlet port 258 of the second assembly.

The magnetic coupling mechanism is visible in all three views (FIGS. 6-8). The assemblies (210, 250) include complementary magnetic coupling elements embedded within their respective housings. The first assembly 210 includes a first magnetic ring 230 circumferentially disposed about the first assembly. Similarly, the second assembly 250 includes a second magnetic ring 270 circumferentially disposed about the second assembly. The magnetic rings (230, 270) are oriented with opposite polarities to create an attractive force when the assemblies are brought into proximity.

When transitioned to the disconnected state, as illustrated in FIG. 7, the withdrawal of the protrusion 244 allows the first one-way valve 242 to return to its sealed position, while the second one-way valve 272 maintains its normal operating position. Both valves work in conjunction with fluid pressure and their inherent material bias to prevent unwanted fluid flow. The first one-way valve 242 prevents fluid discharge from the pump side of the system, while the second one-way valve 272 prevents backflow from the patient side. The magnetic coupling is engineered to separate when a predetermined tensile force is applied to either the coupling 200 or tube 34, providing a reliable safety release mechanism that protects both the patient and the feeding system from damage.

The magnetic coupling configuration provides consistent separation force that can be precisely controlled through proper selection of magnetic ring materials and dimensions. The circumferential arrangement of the magnetic rings (230, 270) ensures both proper valve engagement and uniform separation characteristics regardless of the direction of applied force.

Various features and advantages are set forth in the following claims.

What is claimed is:

1. A coupling comprising:
a plunger assembly comprising:
an inlet;
a plunger chamber in fluid communication with the inlet;
a stem chamber in fluid communication with the plunger chamber; and
a plunger that moves from a first position to a second position;
wherein, the plunger prevents the movement of a fluid between the plunger chamber and the stem chamber when in the first position;
a valve assembly comprising:
a valve chamber with a valve and a rib disposed within; and
an outlet in fluid communication with the valve chamber;
wherein, the coupling moves between a connected state, where the two assemblies are joined together, and a disconnected state, where the two assemblies are disconnected;
wherein, when the coupling is in its connected state, the rib retains the plunger in its second position, the stem chamber is in fluid communication with the valve chamber, and the fluid is able to flow between the inlet and the outlet; and
wherein, when the coupling is in its disconnected state, the fluid biases the plunger in its first position, preventing the flow of the fluid between the plunger chamber and the stem chamber.

2. The coupling of claim 1, wherein the plunger assembly further comprises a gasket disposed within a channel, that fluidically seals the two assemblies when the coupling is in its connected state.

3. The coupling of claim 1, wherein the coupling further comprises a set of enteral feeding connectors respectively positioned at the inlet and outlet.

4. The coupling of claim 1, wherein the plunger further comprises a head and stem, wherein, the head is at least partially retained within the plunger chamber and the stem is at least partially retained within the stem chamber.

5. The coupling of claim 1, wherein the valve further comprises a diaphragm and slit, wherein, the plunger assembly that pierces the diaphragm and retain the valve in an open position.

6. The coupling of claim 4, wherein the plunger further comprises a cutout sized and allows the fluid to pass along the stem.

7. The coupling of claim 1, wherein the plunger assembly further comprises a plunger outer housing and the valve assembly further comprises a valve outer housing, wherein the plunger outer housing is partially disposed within the valve outer housing when in the connected state.

8. The coupling of claim 1, wherein the plunger chamber comprises a first inner surface and a second inner surface forming circular faces of the chamber, and wherein the first inner surface and the second inner surface each comprise a respective circular hole.

9. The coupling of claim 8, wherein the plunger abuts against the second inner surface in the first position to prevent the fluid flow.

10. The coupling of claim 1, wherein the valve chamber comprises a first portion having a first diameter and a second portion having a second diameter different from the first diameter, the first and second portions being connected by a conical transitional section.

11. The coupling of claim 1, wherein the rib bisects the valve chamber and is sized to allow the fluid to flow past the rib toward the outlet.

12. The coupling of claim 1, wherein the plunger assembly and valve assembly are formed from a material selected from the group consisting of resin, plastic, metal, and composites.

13. The coupling of claim 1, wherein the plunger assembly and valve assembly automatically separate upon application of a predetermined force to prevent patient harm.

14. The coupling of claim 1, wherein the valve automatically closes when the coupling transitions to the disconnected state to prevent unwanted fluid discharge.

15. A coupling comprising:
a first assembly comprising:
an inlet port;
a first valve chamber housing a first one-way valve formed of a compliant material; and
a first magnetic ring circumferentially disposed about the first assembly;
a second assembly comprising:
an outlet port;
a second valve chamber housing a second one-way valve;
a protrusion extending from the second assembly toward the first assembly; and
a second magnetic ring circumferentially disposed about the second assembly;
wherein, the coupling moves between a connected state, where the two assemblies are magnetically joined together, and a disconnected state;
wherein, when the coupling is in its connected state, the protrusion mechanically engages with and displaces the first one-way valve creating a continuous fluid pathway between the inlet port and the outlet port; and
wherein, when the coupling is in its disconnected state, the first one-way valve automatically returns to a sealed position preventing fluid discharge.

16. The coupling of claim 15, wherein the first and second magnetic rings are oriented with opposite polarities to create an attractive force when the assemblies are brought into proximity.

17. The coupling of claim 15, wherein the first valve chamber and second valve chamber are generally cylindrical in shape with diameters sufficient to accommodate their respective valves.

18. The coupling of claim 15, wherein the first one-way valve is oriented to permit fluid flow toward the second assembly while preventing backflow toward the inlet port, and the second one-way valve is oriented to prevent backflow from the outlet port.

19. The coupling of claim 15, wherein the magnetic rings separate when a predetermined tensile force is applied to prevent patient harm.
